(19) United States
(12) Patent Application Publication
(10) Pub. No.: US 2009/0087499 A1
(43) Pub. Date: Apr. 2, 2009

(54) DEPILATORY PRODUCT

(76) Inventor: David S. Ott, JR., Easton, PA (US)

Correspondence Address: SCHWEITZER CORNMAN GROSS & BONDELL LLP, 292 MADISON AVENUE - 19th FLOOR, NEW YORK, NY, 10017, US

(21) Appl. No.: 12/111,314

(22) Filed: Apr. 29, 2008

Related U.S. Application Data
(60) Provisional application No. 60/997,005, filed on Sep. 28, 2007.

(51) Int. Cl. A61K 33/00; A61K 31/19

(52) U.S. Cl. 424/722; 514/557

(57) ABSTRACT

The invention comprises a depilatory product based on a complexing gel. The product will remove the hair via reduction and hydrolysis as do chemical depilatories. Despite the peeling nature of the product, the hair is not removed by pulling it out like a wax depilatory. The product is to be applied as a single step from a component that allows the mixing of two separate parts during application to the skin. The final gel is plastic and pliable but non-mobile similar to silicone rubber. The gel should possess strength so that it can be removed from the skin as a single piece without breaking or ripping.

### RELATED APPLICATIONS

This application is related to and claims the priority of provisional application Ser. No. 60/997,005, filed Sep. 28, 2007.

### PRIOR ART

Currently on the market there are many different types of chemical depilatories. They range in form from creams to gel to aerosol mousses and spray products. The main draw back to these products is the required clean up. All chemical depilatories contain a strong alkies usually a metal hydroxide. In addition there is usually a reducing agent used. These chemicals can cause considerable damage to the skin is not properly removed. Likewise they will quickly cause blindness if they get into the eyes. Product wiped off the skin is still active and will corrode aluminum and many organic materials, creating the possibility of property damage or injury to the user, children or pets through inadvertent contact.

### SUMMARY OF INVENTION

The present product will help to reduce these risks. This is because the dangerous chemicals will be trapped in a solid matrix after use simplifying disposal, there will not be liquid which can stick to surfaces. The remaining risks would arise via ingestion of the solid particles or the chemicals leaching from the matrix in water. Both of these risks exist with the liquid products as well.

Another draw back to the chemical depilatories is the great smell usually associated with, the reducing agent used. These almost always contain sulphur, which is difficult to cover up even with the use of masking agents and strong fragrance. The solid matrix of the envisioned product helps to abate the odor by its rubbery nature which traps odors internally.

The purpose of the present product combines the two most popular non-shaving methods of depilation. The product is to form a dry rubbery film on the skin which after a period of a few minutes can be peeled in one piece from the skin. In this manner it is similar to a hot wax depilation. After removal nothing save a very slight residue is left behind, which could be removed with a damp towel. However unlike wax the hair will have been digested by chemicals and not pulled out. Further there is the elimination of a great deal of mess.

It is possible that the present product may help to eliminate some of the associated irritation of chemical depilatories. This is again through the solid matrix reducing the interaction of the active ingredients with the skin. The hair is buried within the matrix, while the skin has only a thin film of contact with the active ingredients.

Compared to waxing this new product has one great advantage. This advantage is lack of pain associated with hair removal. Waxing works by pulling the hair out from the roots. The hair follicle is surrounded by a small muscle that allows the hair to be erected in response to cold or excitement. Also attached with this muscle are nerve endings.

These structures make pulling the hair our fairly hard and painful. The pain creates a fear associated with the waxing procedure which will cause many people not to repeat the procedure. Even if the person does repeat the waxing, the fear can cause the muscles the tense up creating even more pain in subsequent procedures.

The present product will be a great breakthrough in ease and convenience giving added value to the consumer.

### BRIEF DESCRIPTION OF THE INVENTION

There are several key components to the present composition denoted below.

Components (1)-(7) are considered essential while components (8) and (9) are optional.

It has been determined that there are several key components to this product.
1) A solvent phase (water based)
2) A polymer/s capable of creating a hydrosol and complexing to form a hydrogel
3) Depilatory active ingredients
4) Complexing agent/active ingredient delivery system
5) A filler and/or gel strengthening agents
6) A skin protecting agent
7) Odor masking agents and fragrance
8) Chelating materials
9) Colorant materials

Unless otherwise indicated, all percentages of components are on a weight percentage basis, as shown below.

| | | Broad Range | Preferred Range |
|---|---|---|---|
| (1) | Solvent phase (water) | 20-80 | 35-65 |
| (2) | Polymer phase | 0.5-25 | 3-13 |
| (3) | Depilatory Ingredients | 2-25 | 8-15 |
| (4) | Complexing Agent/Active | 0.01-5 | 1-3 |
| | Ingredient | | |
| (5) | Filters/Gel Strengthening Gel | 0.5-25 | 5-12 |
| (6) | Skin protecting Agents | 0.5-2.0 | 1-2 |
| (7) | Chelating Agent | 0.05-0.5 | 0.1-0.2 |
| (8) | Colorant materials | 0.001-3.0 | 0.001-1.0 |
| (9) | Odor masks and fragrances | 0.05-5.0 | 0.5-1.5 |

(Percentages of optional agents (8) and (9) are not required).

### The Solvent Phase

This is the bulk of the product percentage. It should consist mostly water to assure good dissolution of active ingredients. Low MW glycols and alcohols should be avoided due to the increased irritation possible from their disruption of the skins barrier function. This is especially important with the depilatory active ingredients. Also any ingredient sensitive to alkaline hydrolysis must be avoided unless the hydrolysis end products are favorable to the product function and non-irritating. This phase will contain the polymers and possibly some of the active depilatory agents.

### The Polymer/s

The invention contains a polymer which is able to complex or crosslink upon treatment with a suitable compound. The effect of this complexation or crosslinking creates a hydrogel from the hydrosol of the polymer. The hydrogel is tough and resistant to breakage, allowing the gel to be removed in one piece from the skin. The most preferred polymer for use has been found to be polyvinyl alcohol. There are several grades of polyvinyl alcohol available. They differ in the degree of hydrolysis performed on the polyvinyl acetate starting stock. Most commercial grades vary between 85-99%.

### Depilatory Active Ingredients

The active ingredients chosen are as follows:

Is used in order to denature the proteins in the hair causing it to swell by disrupting the hydrogen bonding in the protein chain. This disruption of the hydrogen bonding will open the protein ‘up for easier attach of the peptide bonds by hydroxide ions.
Thioglycolate—This is the chosen reducing agent, and is most commonly used one because of its relative safety compared to thiourea or thioethanol. The reducing agents reduce the disulfide bonds in keratin. Breaking these bonds further opens the protein chain for hydrolysis. Preferably the thioglycolate will be delivered by the product in a salt form. Most preferably the salt will have limited solubility in aqueous medium.
Metal Hydroxide—These act as the OH⁻ donor for the hydrolysis of the protein. Any (M)(OH)x will work where (M) is sodium, lithium, potassium, or calcium. Most preferably calcium will be used due to its limited solubility which causes it to be less irritating to the skin. The preferred pH of the product is between 12 and 14, most preferred pH of 12-12.75.

### Complexing Agent/Active Ingredient Delivery System

In order to have a stable product in the container all of the ingredients cannot be placed together. Over time cations will cause the polyvinyl alcohol PVOH to complex and turn to rubber. Because of this the product must be split into two separate phases that are kept separated until used.

The first is the aqueous solvent phase. This phase will contain the polymer, urea because there are no interactions between the two, humectants, emulsifiers/surfactants and structural agents and an encapsulated complexing agent.

The second is an oil phase which contains the active ingredients. This phase is critical in its formulation. It is important to have readily emulsified oils or esters making up the bulk of the material of this phase. Cyclic silicones, silicone elastomers, propoxylated and ethoxylated esters function very well in this phase if they are not overly hydroscopic or hydrophilic such that they dissolve the active ingredients in the phase. It is important that the ingredients are just suspended in the oil phase with no interaction or dissolution. Most preferably a mixture of cyclic silicones and silicone elastomers is used to suspend the ingredients.

Also included in this phase are emulsifiers, preferably liquid. The formula does not need to create a long term stable emulsion, just one stable to high electrolyte for a few hours to days. In theory this phase should carry the active ingredients until mixed with the solvent phase, upon which it will self emulsify and allow the thioglycolates and hydroxides to dissolve in the water. Once dissolved the actives will begin the process of digesting the hair. The most preferred embodiment of the invention incorporates ethoxilated lauryl alcohol.

The complexing agent is also carried by this phase but must be further encapsulated. If it is not it will react too quickly and not allow proper mixing and dispersion before gelling. Several different encapsulates including cellulose, enteric coating and combinations may be used. The most preferred coat is stearic acid. We made this by melting stearic acid and rapidly stirring the finely crushed complexing into it. While to borax was suspended in the melted stearic acid the mixture was sprayed at high pressure in the same manner as spray drying. This could also be suspended in the aqueous solvent phase which is kept at a lower pH of about 5. At this pH there is little dissociation and solvation of the stearic acid. When combined with the high pH hydroxides of the oil phase when mixed into the aqueous medium the stearic acid slowly salts and dissolves. This occurs at a slow rate due to the solid waxy nature of the stearic acid at ambient temperature. The most preferred complexing agent is borax.

### Fillers/Gel Strengthening Agents

Materials are added to the formula to increase the strength of the set hydrogel. These have been a variety of materials. One of the most common and useful is kaolin clay. Kaolin is low in free cations.

Silica has been used to give the gel strength. Embodiments have included fumed silica and surface treated silicas.

Other agents can be used to control the properties of the hydrosol before gelling or to suspend the powder ingredients. These can be other polymers such as Carbomer or Xanthan gums. Pure viscosity control could be achieved through the use of non-ionic celluloses such as HEC or CMC. These will hydrolyze in the final mixture but may provide temporary needed viscosity control.

Specialty polymers that thicken on increased pH such as Stutture 2001 from national starch may be of benefit.

### Skin Protecting Agent

As with all depilatories a skin protecting agent should be added to the formula. Most preferred is to use dimethicone for this function. It provides and occlusive barrier that can help to protect the skin from the caustic active ingredients. 0.5-2.0% should be sufficient. It is important to not use excessive amounts of a skin protestant like dimethicone or petrolatum because they can coat the hair preventing attach of the protein chain.

### Odor Masks and Fragrances

Although not necessary to the formulation special attention must be paid to the fragrances. Most fragrances are not stable to high pH systems. Some fragrances at this pH seem to cause a crosslinking or complexing with the polyvinyl alcohol because on their addition the polymer phase quickly turns to rubber.

### Chelating Material

The addition of ethylenediaminetetraacetic acid (EDTA) is vitally important because thioglycollates will complex with any free iron and turn a purple color. EDTA salts should be added to both phases.

### Colorants

Colorants are not necessary to the formulation but may be used for commercial appeal.

### Dispensing Container

The dispensing container must be capable of holding and thereafter dispensing the two phases of the product in the correct proportions. Most preferably the dispenser will have a disposable mixing tip to facilitate the complete mixture of the phases before the application to the skin.

In one embodiment, a double plunger system is used. In another dual product chambers in combination with a pump system is employed.

### SPECIFIC EXAMPLE

Formula for preferred embodiment of invention:

| Part A Contains | | |
|---|---|---|
| Deionized Water | Any | QS to |
| Laponite XLS | Southern. Clay | 00.50% |
| | Products | |
| Urea USP | Ruger Chemical | 12.50% |
| Disodium EDTA | Ruger Chemical | 00.10% |
| Cevol 165SF polyvinyl alcohol | Celanese | 07.50% |
| Cevol 523S polyvinyl alcohol | Celanese | 02.50% |
| Keltrol CG Xanthan Gum | CP Kelco | 00.10% |
| Germabin II propylene glycol, | ISP | 01.00% |
| diazolidinyl urea | | |
| methylparaben, propylparaben | | |
| Kaolin 2457 | MPSI | 12.00% |
| Part B Contains: | | |
| DC 9045 dimethicone crosspolymer | Dow Corning | 42.45% |
| DC 245 cyclopentasiloxane | Dow Corning | 25.00% |
| DC 200 350cts dimethicone | Dow Corning | 00.50% |
| Laureth-7 | Protameen | 02.00% |
| Calacium Hydroxide | Ruger Chemical | 09.00% |
| Calcium Thioglycolate | Rona (EMD) | 21.00% |
| Tetrasodium EDTA | Ruger Chemical | 00.05% |

Whereas particular embodiments of the present invention have been described for the purposes of illustration, variations may suggest themselves to those skilled in the art without departing from the invention as claimed.

1. A chemical depilatory which avoids problems of current products such as odor, residue on the skin and pain associated with hair removal which comprises in combination:
(1) Solvent phase
(2) Polymer phase
(3) Depilatory ingredients
(4) Complexing agent/active ingredient
(5) Fillers/gel strengthening agents
(6) Skin conditioning agent

2. The chemical depilatory of claim 1, which is formulated on a weight basis as follows:

| | | Broad Range |
|---|---|---|
| (1) | Solvent phase | 20-80 |
| (2) | Polymer phase | 0.5-25 |
| (3) | Depilatory ingredients | 2-25 |
| (4) | Complexing agent/active ingredient | 0.01-5 |
| (5) | Fillers/gel strengthening agents | 0.5-25 |
| (6) | Skin conditioning agent | 0.5-2.0 |

3. The composition of claim 1 wherein said solvent phase is mostly water to assure good dissolution of active ingredients.

4. The composition of claim 1 wherein the polymer is polyvinyl alcohol.

5. The composition of claim 1 wherein the depilatory ingredient is selected from the group consisting of thiolycolate and metal hydroxides.

6. The composition of claim 1 wherein the complexing agent/active ingredient delivery system comprises (1) an aqueous phase containing a polyvinyl alcohol polymer, and (2) an oil phase containing active ingredients including readily emulsified oils or esters, emulsifiers and complexing agents.

7. The composition of claim 6 wherein the emulsified oil or ester is a mixture of cyclic silicones and silicone elastomers.

8. The composition of claim 1 wherein said emulsifier incorporates ethoxilated lauryl alcohol.

9. The composition of claim 1 wherein the skin conditioning agent is dimethicone.

10. The composition of claim 1 wherein the skin chelating agent is EDTA salts.

11. A chemical depilatory which avoids problems of current products such as odor, residue on the skin and pain associated with hair removal which comprises:
(1) An aqueous phase
(2) Polyvinyl alcohol polymer
(3) A depilatory selected from a group consisting of thioglycolate and metal hydroxides
(4) An oil phase containing active ingredients, including readily emulsified oils or esters, emulsifiers and complexing agents
(5) EDTA as a chelating material
(6) Skin conditioning agents such as dimethicone
